(19) United States
(12) Patent Application Publication
(10) Pub. No.: US 2003/0170278 A1
(43) Pub. Date: Sep. 11, 2003

(54) Microreactor and method of determining a microreactor suitable for a predetermined mammal

(76) Inventors: David E. Wolf, Sudbury, MA (US); Claire A. McGrath, West Boylston, MA (US); Willem M. Kuhtreiber, Shrewsbury, MA (US); Jeanine Lorusso, Sterling, MA (US); Angela D. Pollard, Westminster, MA (US)

Correspondence Address: Allison Johnson, Allison Johnson, P.A., 6016 Logan Avenue South, Minneapolis, MN, 55419, US

(21) Appl. No.: 10/095,503

(22) Filed: Mar. 11, 2002

(51) Int. Cl. A61K 9/00; A61K 9/48; A61K 9/50

(52) U.S. Cl. 424/400; 424/451; 424/93.7

(57) ABSTRACT

A method of determining a suitable microreactor for a predetermined host including identifying a suitable microreactor diameter, optionally identifying a minimum second matrix thickness, and optionally identifying a microreactor having a centrally located microcapsule.

A microreactor having a diameter such that, when implanted in a higher order mammal, the microreactor exhibits no greater than 25% fibrosis for at least 14 days after implantation.

A microreactor that includes a semipermeable membrane capable of eliciting a fibrotic response from a higher order mammal, and a second matrix surrounding the semipermeable membrane, the second matrix being of a thickness sufficient to prevent nucleation of fibrosis by the semipermeable membrane when the microreactor is implanted in a higher order mammal for at least 14 days.

### BACKGROUND OF THE INVENTION

The invention relates to determining a suitable microreactor for a predetermined mammal.

Various research efforts have been directed to implanting encapsulated living cells and tissues in mammals with the goal of having the living cell or tissue perform a function in the mammal. Typically the encapsulated cells are implanted in a mammal that either lacks the living cells, or possesses the cells, but the cells are functioning at a less than the desired level. Encapsulated pancreatic islet cells, for example, have been implanted in diabetic mammals in an attempt to restore the mammal's glucose-responsive insulin function.

The living cells encapsulated in the microcapsules are often allogeneic cells (i.e., cells from donors of the same species as the host) or xenogeneic cells (i.e., cells from a species that differs from that of the host). As a result such microcapsules often come under immune attack when implanted in a host's peritoneum. One major pathway of immune attack is the proinflammatory immune response, which results in a walling off of the encapsulated cell through the formation of a fibrous sheath at the surface of the microcapsule. As the amount of fibrosis on the surface of the microcapsule increases, the flow of nutrients to the living cells within the microreactor decreases. Eventually the amount of fibrosis surrounding the microcapsule is so great that nutrients cannot pass through to the microcapsule to the cells, waste products cannot pass out of the microcapsule, and the cells die. The fibrotic cells also can secrete lytic factors. Once the cells die the microreactor can no longer perform the intended function.

Encapsulated pancreatic islet cells have been used in an attempt to restore glucose homeostasis in a diabetic patient. True glucose homeostasis in which the moment-to-moment fine regulation of glucose levels that is performed by the pancreas of a non-diabetic person is very difficult or impossible to achieve in diabetic patients with conventional subcutaneous or pulmonary administration of insulin. The failure to achieve true glucose homeostasis can lead to serious secondary complications including nephropathy, retinopathy, micro- and macrovascular disorders, heart disease and stroke. It has been shown that improved blood glucose control through intensive insulin therapy can reduce these secondary complications. However, methods of restoring physiological control of glucose levels are necessary in order to achieve true glucose homeostasis in a diabetic patient. It would be desirable to achieve true glucose homeostasis using microreactors, however, the fibrosis that occurs on the microreactors has been a huge impediment to successfully achieving that goal.

### SUMMARY

In one aspect, the invention features a microreactor that includes a) a microcapsule having an average diameter greater than 580 μm, the microcapsule including a first matrix and a living agent disposed in the first matrix, b) a semipermeable membrane surrounding the microcapsule, and c) a second matrix surrounding the semipermeable membrane, the average distance from the semipermeable membrane to the exterior surface of the second matrix being at least 600 μm. In one embodiment, the microcapsule is essentially centrally located in the second matrix. In another embodiment, the average distance from the semipermeable membrane to the exterior surface of the second matrix is at least about 700 μm, at least about 800 μm or at least about 1000 μm. In other embodiments, the microreactor has an average diameter of at least about 2000 μm, at least about 3000 μm, at least about 4000 μm, or at least about 5000 μm. In some embodiments, the microcapsule has an average diameter of at least about 600 μm. In other embodiments, the microcapsule has an average diameter of at least about 700 μm, at least about 800 μm, at least about 1000 μm, at least about 2000 μm or at least about 3000 μm.

In another embodiment, the living cell includes an islet cell. In some embodiments, the living cell is selected from the group consisting of porcine islet cells, canine islet cells, bovine islet cells, ovine islet cells, human islet cells, non-human primate islet cells, and combinations thereof.

In some embodiments, the semipermeable membrane includes polyamino acid. The polyamino acid can be selected from the group consisting of polylysine, polyornithine, polyarginine, polyhistidine and combinations thereof.

In another aspect, the invention features a method of treating a mammal that includes implanting at least one microreactor in the mammal, the microreactor including a) a microcapsule that includes a first matrix and a living agent disposed in the first matrix, the microcapsule having an average diameter greater than 580 μm, b) a semipermeable membrane surrounding the microcapsule, and c) a second matrix surrounding the semipermeable membrane, the average distance from the semipermeable membrane to the exterior surface of the microcapsule being at least 600 um. In one embodiment, the implanted microreactor is free of an amount of fibrosis lethal to the living agent for at least 14 days. In another embodiment, the implanted microreactor exhibits no greater than 25% fibrosis for a period of at least 14 days. In other embodiments, the implanted microreactor exhibits no greater than 20% fibrosis for a period of at least 14 days. In some embodiments the implanted microreactor exhibits no greater than 10% fibrosis for a period of at least 14 days.

In other aspects, the invention features a microreactor that includes a) a microcapsule that includes a first matrix and a living agent disposed in the first matrix, b) a semipermeable membrane surrounding the microcapsule, and c) a second matrix surrounding the semipermeable membrane, the microreactor, when implanted a higher order mammal for at least 14 days, exhibiting no greater than 25% fibrosis. In one embodiment, the mammal is a human being. In another embodiment, the mammal is selected from the group consisting of dogs, cats, horses, cows, pigs, and nonhuman primates. In one embodiment, the microcapsule has an average diameter of at least 580 μm. In another embodiment, the microreactor has an average diameter of at least 1500 μm. In some embodiments, the semipermeable membrane includes polyamino acid. In other embodiments, the semipermeable membrane includes poly-L-lysine, polyornithine, polyarginine, polyhistidine or a combination thereof. In some embodiments, the microreactor, when implanted in a higher order mammal for at least 14 days, exhibits no greater than 20% fibrosis. In other embodiments, the microreactor, when implanted in a higher order mammal for at least 14 days, exhibits no greater than 10% fibrosis.

In another aspect, the invention features a microreactor that includes a microcapsule, a semipermeable membrane surrounding the microcapsule, the semipermeable membrane including a component capable of eliciting a fibrotic response from a higher order mammal, and a second matrix surrounding the semipermeable membrane, the second matrix being of a thickness sufficient to prevent nucleation of fibrosis by the semipermeable membrane when the microreactor is implanted in a higher order mammal for at least 14 days. In one embodiment, the mammal is a human being. In embodiment, the mammal is selected from the group consisting of dogs, cats, horses, cows, pigs, and non-human primates.

In other aspects the invention features a method of identifying a microreactor suitable for implantation in a predetermined mammal, the method including implanting at least one microcapsule in a plurality of the predetermined mammal, the average diameter of the at least one microcapsule implanted in the mammals of one set of mammals being different from the average diameter of the at least one microcapsule implanted in the mammals of another set of mammals, explanting the microcapsules from the mammals after a period predetermined to be sufficient to elicit a fibrotic response, analyzing the amount of fibrosis present on the surface of the explanted microcapsules, determining whether the microcapsules of a set exhibit an average of no greater than 25% fibrosis, if such a microcapsule is present, identifying the average diameter of the microcapsule as suitable for implantation in the mammal, and if such a microcapsule is not present, repeating the implanting, explanting, analyzing and determining until a microcapsule exhibiting no greater than 25% fibrosis is present, and identifying the average diameter of the microcapsule. In some embodiments, the determining includes determining whether at least one microcapsule exhibits no greater than 20% fibrosis. In other embodiments, the determining includes determining whether at least one microcapsule exhibits no greater than 10% fibrosis. In another embodiment, the determining includes determining whether at least one microcapsule exhibits no greater than 5% fibrosis.

In one embodiment, the volume of microcapsules implanted in each mammal is essentially the same. In other embodiments, the total surface area of the microcapsules implanted each mammal is essentially the same.

In other aspects, the invention features a method of treating a mammal that includes implanting a microreactor having a predetermined diameter in the mammal, the microreactor diameter having been predetermined for the mammal according to a method described herein.

In another aspect, the invention features a method of identifying a microreactor suitable for implantation in a predetermined mammal, the method including implanting at least one microreactor in a plurality of the predetermined mammal, the microreactor including a microcapsule, a semipermeable membrane surrounding the microcapsule and a second matrix surrounding the semipermeable membrane, the composition of the semipermeable membrane being capable of eliciting a fibrotic response from the predetermined mammal, the second matrix thickness of the microreactor implanted in the mammals of one set of mammals being different from the second matrix thickness of the microreactors implanted in the mammals of another set of mammals, explanting the microreactors from the mammals after a period predetermined to be sufficient to elicit a fibrotic response from the predetermined mammal, determining whether a microreactor is free of nucleated fibrosis, and, if a microreactor is free of nucleated fibrosis, identifying the thickness of the second matrix of the microreactor as suitable for implantation in the predetermined mammal, if a microreactor exhibits nucleated fibrosis, repeating the implanting, explanting and determining until a second matrix thickness suitable for implantation in the predetermined mammal is identified.

In another aspect, the invention features a method of treating a mammal that includes implanting a microreactor in a mammal, the microreactor including a second matrix having a thickness predetermined for the mammal according to a method described herein. In one embodiment, the mammal is a human being. In other embodiments, the microreactor includes a microcapsule, a semipermeable membrane surrounding the microcapsule, and a second matrix surrounding the semipermeable membrane. In another embodiment, the microcapsule is substantially centrally located in the microreactor. In some embodiments, the mammal is diabetic, the method further including implanting the microreactors in an amount sufficient to achieve glucose homeostasis in the mammal. In one embodiment, the mammal is diabetic and dependent upon a source of insulin exogenous to the mammal, the method further including implanting the microreactors in an amount sufficient to achieve a reduction in the mammal's dependence on exogenous insulin.

In other aspects, the invention features a method of identifying a microreactor suitable for implantation in a predetermined mammal, the microreactor that includes a microcapsule, a semipermeable membrane surrounding the microcapsule and a second matrix surrounding the semipermeable membrane, the method including determining a suitable average microreactor diameter and determining a suitable second matrix thickness.

In another aspect, the invention features a microreactor suitable for implantation in a predetermined mammal, the microreactor including a microcapsule that includes a matrix and a living agent disposed in the matrix, a semipermeable membrane surrounding the microcapsule, and a second matrix surrounding the semipermeable membrane, the suitability of the microreactor having been predetermined for the mammal according to a method described herein.

In other aspects, the invention features a method of selecting a microreactor for implantation in a mammal, the method including a) identifying a microreactor that includes a microcapsule including a first matrix, a living agent disposed in the first matrix, and a semipermeable membrane surrounding the first matrix, and a second matrix surrounding the semipermeable membrane, the microcapsule being essentially centrally located in the second matrix, the average distance between the exterior surface of the microcapsule and the exterior surface of the second matrix being at least 600 μm, and b) removing the microreactor from a plurality of microreactors. In one embodiment, the microreactor is removed from a plurality of microreactors prior to identifying the microreactor. In other embodiments, the microreactor is removed from the plurality of microreactors subsequent to identifying the microreactor.

The invention provides the ability to identify a suitable microreactor diameter for any mammal. Once the suitable microreactor diameter is identified for a particular mammal, a microreactor of the predetermined diameter can be implanted in such mammals without eliciting a fibrotic response that would prevent the microreactor from providing a therapeutically effective amount of therapeutic substance to the host.

The invention also provides the ability to identify a suitable microreactor second matrix thickness for any mammal. Once the second matrix thickness has been identified for a particular mammal, a microreactor having the predetermined second matrix thickness can be implanted in such mammals and remain free of nucleated fibrosis during implantation.

Other features and advantages will be apparent from the following description of the preferred embodiments and from the claims.

### BRIEF DESCRIPTION OF THE DRAWINGS

FIG. 1 is a color photograph of a microreactors exhibiting nucleated fibrosis.

FIG. 2 is a plot of % viability versus % fibrosis.

FIG. 3 is a color photograph of a microreactor of Example 1.

FIG. 4 is a plot of blood glucose level (mg/dL) versus time.

FIG. 5 is a plot of % fibrosis versus microreactor diameter (μm).

FIG. 6 is a plot of % fibrosis versus microreactor diameter (μm).

### DETAILED DESCRIPTION

The present inventors have discovered that the diameter of an implanted microreactor has an effect on the fibrotic response mounted by a host against the microreactor and that the microreactor diameter can be optimized to minimize the host's fibrotic response to the microreactor. The present inventors have also discovered that the proximity of a fibrogenic semipermeable membrane to the exterior of the microreactor correlates to the nucleation of fibrosis at the surface of the microreactor, and there is a minimum thickness of matrix surrounding the semipermeable membrane necessary to prevent fibrosis from nucleating at the surface of an implanted microreactor.

Suitable Microcreator

The microreactor includes a microcapsule that includes a first matrix and a living agent disposed in the first matrix, a semipermeable membrane surrounding the microcapsule and a second matrix surrounding the semipermeable membrane. Preferably the microcapsule is centrally located in the second matrix of the microreactor.

The microreactor has a diameter that allows the microreactor, when implanted in a host, to remain free of an amount of fibrosis that would prevent the microreactor from providing a therapeutically effective amount of therapeutic substance to the host. The microreactor is preferably constructed to remain free of a lethal amount of fibrosis, i.e., an amount of fibrosis that is capable of killing the living agent, prior to the end of the period over which the living agent is to provide a therapeutic effect to the host. Useful microreactors have a diameter, i.e., the average cross-sectional dimension through the center of the microreactor, that is at least about 1500 μm, at least about 2000 μm, at least about 3000 μm, at least about 4000 μm, or at least about 5000 μm. When the host is a mouse, for example, a suitable diameter is greater than 2000 μm.

The matrix surrounding the semipermeable membrane, i.e., the second matrix, is of a thickness sufficient to prevent nucleation of fibrosis by the semipermeable membrane. The second matrix thickness also is preferably uniform around the circumference of the microcapsule such that the microcapsule is centrally located in the second matrix. Preferably the thickness of the second matrix is the minimum thickness that will prevent nucleated fibrosis. Useful microreactors have second matrix thickness of at least 600 μm, at least 700 μm, at least 1000 μm or at least 1500 μm. Microreactors that are to be implanted in mice preferably have a second matrix thickness of at least 600 um. When more than one microcapsule is present in the microreactor, there is preferably at least 600 μm of second matrix between the semipermeable coating of the microcapsules and the host. The additional microcapsules can be of a variety of configurations and dimensions.

The microreactor preferably includes a single microcapsule having an average diameter of greater than 580 μm. Useful microreactors include a microcapsule having a diameter of at least about 600 μm, at least about 700 μm, at least about 800 μm, at least about 1000 μm, at least about 2000 μm or at least about 3000 μm.

The microreactor includes at least one living agent. The living agent is preferably a living cell, living tissue or a combination thereof. The living cell can be syngeneic, autogenetic, allogeneic or xenogeneic relative to the host. The living cell can be a primary isolate from a donor tissue, an established cell line, genetically engineered cells, or differentiated cells derived from stem cells. Useful living agents include, e.g., islet cells (e.g., human, ovine, bovine, porcine and canine islet cells), tissue culture cells that produce insulin, cells genetically engineered to produce insulin, and insulin producing differentiated cells derived from stem cells. Preferably the living cells are selected to secrete a therapeutically useful substance. Examples of such substances including hormones, growth factors, trophic factors, neurotransmitters, lymphokines, antibodies and combinations thereof. Examples of therapeutic cell products include insulin, nerve growth factors, interleukins, parathyroid hormone, erythropoietin, albumin, transferin, and Factor VIII.

The composition and properties of the matrices of the microreactor are selected to facilitate the function of the particular matrix. The matrix of the microcapsule, i.e., the first matrix, provides physical support to the living agent to allow the living agent to remain suspended in the matrix. The microcapsule matrix also provides an aqueous environment for the living agent and is compatible with the living agent, i.e., it does not kill the living agent. The composition of the matrix that surrounds the microcapsule, i.e., the second matrix, is selected to compatible with and accepted by the host in which the microreactor is to be implanted (i.e., the host will not mount an immune response to the composition of the second matrix).

The microreactor matrices are composed of biocompatible gels. Examples of such gels include hydrogels, i.e., a three-dimensional network of cross-linked hydrophilic polymers. Suitable hydrogels include, e.g., gels that carry a net negative charge (e.g., alginate), gels that carry a net positive charge including, e.g., extracellular matrix components such as collagen and laminin, gels that include a net neutral charge including, e.g., crosslinked polyethylene oxide and polyvinyl alcohol, and agarose. Suitable extracellular matrix components are commercially available under the trade designation MATRIGEL from Collaborative Biomedical (Bedford, Mass.), and VITROGEN from Cohesion Technologies (Palo Alto, Calif.).

The matrices can include other additives including, e.g., substances that support, promote or a combination thereof, the function of the living agent. Examples of such substances include natural and synthetic nutrient sources, extracellular matrix components, growth factors, growth substances, regulatory substances, feeder cells, accessory cells, oxygen carriers (e.g., hemoglobin and fluorocarbons), and combinations thereof.

The semipermeable membrane surrounding the microcapsule allows the passage of substances up to a predetermined size and provides an effective barrier to the passage of substances larger than the predetermined size. The semipermeable membrane preferably has a molecular weight cut off range, i.e., the lowest molecular weight that is allowed to pass through the membrane, sufficient to provide an effective barrier to the movement of cells including, e.g., the living agent, into or out of the microcapsule, and to permit the passage of nutrients, waste and the therapeutic substance secreted by the living agent through the semipermeable membrane. The molecular weight cutoff range is selected to maintain the viability and function of the encapsulated living agent. The molecular weight cutoff range can also be selected based on the type and extent of immunological rejection anticipated for the device after the device is implanted.

In some applications, the molecular weight cutoff range will be selected to permit the passage of a physiological control through the membrane. The physiological control is an agent that is produced by the host to signal a function that the living agent of the microreactor is to provide to the host. In the case where islets are the living agent, glucose and the glucose level in a host can function as physiological controls. Microreactors that include islets preferably have a molecular weight cut off range sufficient to allow insulin to flow out of the microreactor and glucose and other nutrients to flow in to the microreactor, while effectively prohibiting the passage of other moieties such as antibodies.

The molecular weight cut off range is a function of the pore size of the semipermeable membrane. The pore size can be controlled through size exclusion and Nucleopore membrane technologies available from Whatman (Newton, Mass.).

Useful molecular weight cut off ranges include, e.g., a molecular weight cutoff that will effectively prevent cell mediated immune response cells from entering the microcapsule, a molecular weight cutoff that will effectively prevent humoral mediated immune response agents, e.g., IgG, from entering the microcapsule (preferably a molecular weight cut off at least 150,000), and a molecular weight cutoff that will effectively prevent cytokines from entering the microcapsule, preferably a molecular weight cutoff of at least 20,000, more preferably a molecular weight cutoff of at least 10,000.

The semipermeable membrane preferably includes polyamino acid. Useful polyamino acids include, e.g., polylysine, polyornithine, polyalanine, polyarginine and polyhistidine. Other useful semipermeable membranes include, e.g., chitosan, polyacrylonitrile/polyvinylchloride, polyethylene oxide, polyvinyl acetate, polyacrylonitrile, polymethylmethacrylate, polyvinyldifluoride, polyethylene oxide, polyolefins (e.g., polyisobutylene and polypropylene), polysulfones, cellulose derivatives (e.g., cellulose acetate and cellulose butyrate), and combinations thereof. One example of a useful semipermeable membrane composition is poly-L-lysine having a molecular weight less than 15 kDa, preferably from 9 kDa to 13 kDa, more preferably from 9 kDa to 11 kDa.

A semipermeable membrane can also result from modifying a portion of the structure of the microcapsule matrix or a portion of the first matrix. One method of modifying the structure of the microcapsule matrix includes crosslinking the microcapsule matrix using metal ions including, e.g., calcium ions, barium ions, iron ions and combinations thereof. The degree of crosslinking affects the porosity of the resulting membrane.

Method of Determining A Suitable Microreactor

Determining a suitable microreactor for a predetermined host, i.e., mammal, includes identifying a suitable microreactor diameter, optionally identifying a minimum second matrix thickness, and optionally identifying a microreactor having a centrally located microcapsule.

Determining a suitable microreactor diameter for a predetermined host includes implanting at least one microcapsule in a number of the predetermined host, where the diameter of the microcapsules implanted in the sets of hosts differs from one set of hosts to the other sets of hosts. For ease of discussion the average cross-sectional dimension taken through the center of the microcapsule or microreactor will be referred to as the diameter of the microcapsule or microreactor. It is to be understood that the disclosure encompasses and is applicable to microcapsules and microreactors that are of shapes other than spherical and substantially spherical. When a set, i.e., more than one, of microcapsules is implanted in a host, the diameter of the microcapsules of the set is essentially the same.

The microcapsules of the study include a matrix and may include other components including, e.g., a living agent, a semipermeable membrane and a second matrix surrounding the semipermeable membrane. Preferably the volume of implanted microcapsules, the total surface area of implanted microcapsules or a combination thereof, is constant from host to host.

The method further includes explanting the microcapsules after a period of time sufficient for the host to mount a proinflammatory response to a foreign body (e.g., 14 days), analyzing the surface of the microcapsules for fibrosis, determining the average % fibrosis, i.e., the percent of the total surface area of the microcapsule that is covered with fibrosis, present on the microcapsules of each set, and identifying the diameter of those microcapsules that are free of an amount of fibrosis lethal to the living agent. Microcapsules that exhibit no greater than 30% fibrosis are determined to have a microcapsule diameter suitable for implantation in the predetermined host. Preferably the microcapsules have no greater than 25% fibrosis, more preferably no greater than 20% fibrosis, more preferably no greater than 10% fibrosis, most preferably the microcapsules are free of fibrosis. The method is repeated until a suitable diameter is identified.

Where more than one suitable microreactor diameter is identified for a predetermined host, the microreactor having the smallest diameter is preferably selected as the suitable microreactor diameter for the predetermined host.

Once the suitable microreactor diameter has been determined for a particular mammal, microreactors having the predetermined diameter can be selected for implantation in others of the mammal without again performing the method of determining a suitable microreactor diameter.

The method of determining a suitable thickness for the second matrix of the microreactor employs a microcapsule that includes a first matrix (optionally a living agent disposed in the first matrix), a semipermeable membrane surrounding the first matrix, and a second matrix surrounding the semipermeable membrane. The semipermeable membrane for purposes of determining a suitable second matrix thickness includes a composition capable of causing fibrosis when exposed to the host. Poly-L-lysine is one example of such a composition. Preferably the microcapsule selected for the study includes a microcapsule centrally located within a second matrix such that an essentially uniform second matrix thickness surrounds the microcapsule.

The method of determining a suitable thickness for the second matrix of the microreactor for a predetermined host includes implanting at least one microcapsule in a number of the predetermined host, where the thickness of the second matrix of the microcapsules implanted in the hosts of one set of hosts differs from the thickness of the second matrix of the microcapsules implanted in the other sets of hosts. When a set, i.e., more than one, of microcapsules is implanted in a host, the thickness of the second matrix of the microcapsules of a set is essentially the same. When determining a suitable second matrix thickness, preferably at least one of the volume of implanted microcapsules and the surface area of the implanted microcapsules is held constant for each mammal receiving the microcapsules.

The method further includes explanting the microcapsules after a period of time sufficient to elicit a fibrotic response to a foreign body from the host (e.g., 14 days), analyzing the microcapsules for nucleated fibrosis, and identifying the thickness of the second matrix of those microreactors that are free of nucleated fibroses. Microcapsules that include nucleated fibrosis are determined to have an insufficient second matrix thickness to prevent fibrosis from occurring. Microcapsules that are free of nucleated fibrosis are determined to have a second matrix thickness suitable for implantation in the mammal.

The above-described method is repeated using microcapsules in which the thickness of the second matrix differs from that of the previously tested microcapsules until a suitable second matrix thickness is determined.

The number of microcapsules implanted in the hosts, the size ranges of the microcapsules and the number of hosts necessary to identify the appropriate second matrix thickness can be determined based upon experimental design and results obtained during each execution of the method. The number of hosts is defined by laws of statistics (e.g., the statistical technique of Power Analysis) for each of N hosts in which an average value (Nave) of fibrosis is obtained. The Nave values are averaged and the differences between the groups are judged significant if P≦0.02 using the Student's Unpaired T-test.

Once the second matrix thickness has been determined for a particular mammal, a microcapsule having the determined second matrix thickness can be selected for implantation in others of the mammal without repeating the method of determining a suitable second matrix thickness.

Various methods can be used to identify microreactors having centrally located microcapsules including, e.g., visually examining a microreactor, electronically sensing a microreactor and combinations thereof. A microscope or a magnifying camera can aid the visual examination of a microreactor. The presence of a centrally located microcapsule can be sensed electronically using various methods including e.g., weighing the microreactor, impinging light on the microreactor, and combinations thereof. The presence of a microreactor having a centrally located microcapsule can be identified in a volume of microreactors and then removed from the volume for subsequent use. Alternately a microreactor can be removed from a volume of microreactors, analyzed to determine whether a microcapsule is centrally located in the microreactor and, if a centrally located microcapsule is present, selected for subsequent use.

The microreactor can be removed from a volume of microreactors using various techniques including, e.g., hand removal (e.g., with the aid of tools such as a perforated Moria spoon) and mechanical removal using various systems including, e.g., robotic systems. The steps of identifying and removing can also be automated.

The techniques for selecting microreactors that include centrally located microcapsules can also be applied to identify and select microreactors based on the thickness of the second matrix of the microreactor.

Method of Treating

The microreactors can be used to treat a mammal by implanting at least one suitable microreactor in the mammal. The amount of living agent implanted in the mammal is preferably sufficient to provide the desired level of therapeutic substance to the mammal. Microreactors can be optimized for implantation in a variety of mammals including, e.g., mice, rats, and higher order mammals including, e.g., dogs, cats, pigs, goats, horses, cows, human beings and non-human primates including, e.g., apes, monkeys and chimpanzees.

The microreactor provides a therapeutic substance to a host by virtue of the ability of the living agent within the microreactor to manufacture and secrete the therapeutic substance. Preferably a sufficient number of living agents are implanted in the host to provide a therapeutically effective amount of the therapeutic substance to the host. A therapeutically effective amount can be achieved in various ways including by implanting a sufficient number of microreactors in the host or by implanting a single microreactor having a sufficient number of living agents disposed therein to achieve the desired therapeutic effect. The living agent may be an agent that is capable of stimulation by a physiological control.

Other Cell Therapy Applications

A variety of cells can be encapsulated in the microreactor and employed in the methods described herein including, e.g., hepatocytes for the treatment of liver failure and enzymatic defects, adrenal chromaffin cells for chronic pain, genetically-engineered cells that produce clotting factors VIII and IX for hemophilia, human growth factor for dwarfism, erythropoietin for anemia, parathyroid cells for hypocalcemia, nerve growth factors for ALS, and cells for treating Parkinson's disease, Alzheimer's disease epilepsy, Huntington's disease, spinal cord injuries and strokes, and combinations thereof.

The invention will now be described by way of the following examples.

### EXAMPLES

Test Procedures

Test procedures used in the examples include the following.

Sample Preparation

Islet Isolation-Porcine

Islet isolation and purification is performed according to procedures modified from the methods of Warnock, C. L. and R. V. Rajotte, “Critical Mass of Purified Islets that Induce Normoglycemia After implantation into Diabetic Dogs,” Diabetes, vol. 37, p. 467-70 (1988) and Lanza, R. P. et al., “Xenotransplantation of Canine, Bovine, and Porcine Islets in Diabetic Rats Without Immunosuppression,” Proc. Nat'l. Acad. Sci. U.S.A., Vol. 88(24), p. 11100-11104 (1991).

The porcine pancreas is infused via the pancreatic duct with Variant D, UW-D[70] cold organ preservation solution (University of Wisconsin). The gland is excised and infused via the pancreatic duct system with a composition including the UW-D[70] solution and 0.4 g/L collagenase lot H (Crescent Chemical, Hauppage, N.Y., Sevac #1022001). The gland is transported on ice to the laboratory. The pancreas is digested with collagenase at 40° C. Islet purification is performed by density centrifugation on a discontinuous Euroficoll gradient (Sigma-Aldrich, St. Louis, Mo.) having densities of (w/v): 11%, 20.5% and 27%.

Islets are collected from the first interphase (between 11% and 20.5%) and washed several times to remove excess Euroficoll. The number of isolated islets is determined and the purity of the islets is evaluated. The islets are distributed in 20 mm×100 mm suspension culture dishes in an amount of from 60,000 to 80,000 islets/20 mm×100 mm suspension culture. The islets are cultured in a solution of Hams F12 culture media (herein after “media”) (Mediatech, Herndon, Va.), 10% Horse Serum (Hyclone, Logan, Utah) and antibiotics.

Poly-L-Lysine Coating Method

A volume of microcapsules is measured and diluted with a 15 fold excess volume of the desired % Poly-L-Lysine (PLL) solution (e.g., from 0.01% to 0.2% (w/v) PLL) (˜9 kDa #P6516) (Sigma Aldrich, St. Louis, Mo.). The microcapsules are incubated in the PLL solution at 37° C. for three minutes with constant agitation. The PLL coated microcapsules are then immediately filtered, rinsed, and washed three times with media.

Method of Implanting Encapsulated Islets

Mice

Mice are anesthetized by injecting 90 mg/kg body weight (BW) ketamine (Henry Schein Inc., Melville, N.Y.) and 20 mg/kg BW xylazine (Henry Schein) intramuscularly. The surgical field is prepared by shaving the abdomen and then swabbing the abdomen with 70% isopropyl alcohol and iodine. A small medial incision is made through the abdominal skin and muscle into the peritoneal cavity. Encapsulated islets are delivered into the peritoneal cavity via a sterile pipet. The muscle wall is closed using 5.0 vicryl sutures and the skin is stapled or sutured closed.

Test for Glucose Levels after Implantation of Encapsulated Islets

Blood glucose levels are tested using the Accu-Chek Simplicity blood glucose meter (Roche Diagnostics Corp., Indianapolis, Ind.).

Mice

Baseline glucose levels are taken prior to microreactor implant (i.e., pre-implant glucose levels) and then every 2-5 days after implantation for the duration of the implant. The glucose level is determined by removing a sample of blood from the tail vein of a mouse and measuring the glucose level present in the sample using the Accu-Chek Simplicity blood glucose meter.

Method of Explanting Encapsulated Islets and Histological Assays

Euthanasia is performed by carbon dioxide overdose. An abdominal incision is made into the peritoneal cavity to retrieve aseptically the microreactors by lavage. A gross visual examination of the internal organs is performed. Retrieved microreactors are washed with a phosphate buffered saline solution containing calcium ions (Ca⁺⁺) and magnesium ions (Mg⁺⁺) and processed using Neutral Red analysis to determine % fibrosis and % viability.

% Viability Assay

After the microreactors are explanted, they are thoroughly washed with phosphate buffered saline solution containing calcium ions (Ca⁺⁺) and magnesium ions (Mg⁺⁺). The microreactors are incubated in 0.0004% (w/v) Neutral Red 3-amino-7-dimethylamino-2-methyl-phenazine hydrochloride (Sigma-Aldrich, St. Louis, Mo.) for approximately one hour at 37° C., washed two or three times with phosphate buffered saline with Ca⁺⁺ and Mg⁺⁺ and then scored for viability. Encapsulated islets are counted using a microscope and scored by the % Neutral Red incorporation assay. Viability is estimated based on the following categories: 100%, 75%, 50%, 25% and 0%. Reported % viability is calculated by using a weighted average.

% Fibrosis Assay

Microreactors are explanted and then thoroughly washed with a solution of phosphate buffered saline with Ca⁺⁺ and Mg⁺⁺. The microreactors are incubated in 0.0004% (w/v) Neutral Red for approximately one hour at 37° C. and then washed two or three times with a phosphate buffered saline with Ca⁺⁺ and Mg⁺⁺ solution. Each capsule is scored for % fibrosis. This scoring is based on the estimated percent of the capsule surface area that is covered with cellular adhesion. The estimated % fibrosis is determined for each capsule and is assigned to one of the following categories: 100%, 75 %, 50%, 25% and 0%. A weighted average of the % fibrosis on the capsules is calculated and reported.

Histology

Microreactors are examined using standard histology methods. Microreactors are fixed in Bouin's fixative (VWR, Bridgeport, N.J.), dehydrated, embedded in paraffin, sectioned as five micron thick sections and stained with hematoxylin and eosin.

Insulin Secretion Test Method

Insulin levels are analyzed using a Human Insulin ELISA kit 008-10-1113-01 (ALPCO, Windham, N.H.). Blood samples are read at 450 nm using an OptiMax plate reader (Molecular Devices Corp., Sunnyvale, Calif.). Data software reduction is performed using SoftMax Pro, version 2.4.1. Serum insulin levels from porcine implants are determined using porcine insulin standards in the Human Insulin ELISA kit.

Method of Determining In Vitro Glucose Challenge

Explanted microreactors are immediately placed in a culture media that includes Hams F12 and 10% Horse Serum, and washed extensively. All incubations occur in a 5% CO₂ environment at 39° C. These microreactors are placed in Duldeco's modified eagle media (DMEM) #11966-025 (Gibco, Grand Island, N.Y.) supplemented with 50 mg/dL glucose (G50) and allowed to secrete insulin for 4-24 hrs. Samples of the culture media are collected and frozen. Further sampling of 4-6 hr. incubations in media supplemented with G50 are collected to determine baseline insulin levels. Microreactors are then challenged with culture media supplemented with 400 mg/dL glucose (G400) for 6-8 hr. incubations. Subsequent G50 incubations are collected. All Insulin levels are analyzed on Human Insulin ELISA kits # 008-10-1113-01 (ALPCO, Windham, N.H.).

### Comparative Example 1

Alginate spheres (i.e., microcapsules without islets), 650 μm in diameter, were coated with 0.2% (w/v) PLL according to the PLL Coating Method. The PLL-coated 650 μm microcapsules were then added to a solution of approximately 1.7% (w/v) sodium alginate (PRONOVA, Norway) at a ratio of 1:4 (v/v) (microcapsules to alginate). This composition was passed through an 18 G airjet into a 1.5% (w/v) buffered CaCl₂ solution using a syringe pump at a flow rate of from 0.6 ml/min to 0.8 ml/min. Nitrogen gas, flowing at a rate of approximately 3 liters (l)/min was introduced to shear off the droplet at the desired size. The resulting microcapsule-containing droplets were allowed to polymerize in CaCl₂ for four minutes to form 1100 μm diameter microcapsules. The 1100 μm microcapsules were then washed three times with media to remove all traces of CaCl₂.

A volume of the 1100 μm microcapsules was implanted in forty normal nondiabetic C57-BL/6 diabetic mice. Upon removal at 2 weeks, these microcapsules were evaluated for % fibrosis using Neutral Red. The microcapsules viewed under a microscope showed fibrosis levels of 53%±4% (Mean±S.E.M.) (N=40).

### Comparative Example 2

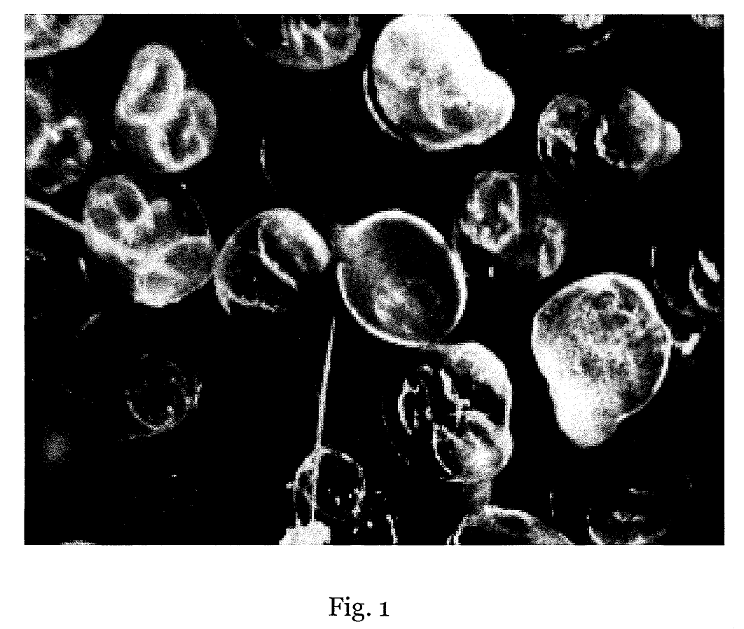

Microcapsules without islets, having a diameter of 1100 μm and prepared according to Comparative Example 1, were implanted in five normal non-diabetic C57BL/6 mice according to the Method of Implanting Encapsulated Islets. The microcapsules were removed at 18 days, stained with 0.0004% (w/v) Neutral Red, and assayed for % fibrosis. The removed microcapsules are shown in FIG. 1. Fibrosis nucleated at sites where the PLL is proximal to the outer surface of the microcapsule.

### Comparative Example 3

Microcapsules having a diameter of 650 μm were prepared by diluting 20,000 porcine islets/ml in an aqueous solution of approximately 1.7% (w/v) sodium alginate and then dropping the composition from a 22 G airjet into 1.5% (w/v) buffered CaCl₂ solution using a syringe pump at a flow rate ranging from 0.6 to 0.8 ml/min. Nitrogen gas, flowing at a rate of 3 l/min, was introduced to shear off the droplet at the desired droplet size. The droplets were allowed to polymerize for three minutes in CaCl₂ solution to form the 650 μm diameter microcapsules. The 650 μm microcapsules are then washed three times with media to remove all traces of CaCl₂.

The 650 μm microcapsules were then coated with 0.2% (w/v) PLL.

The PLL-coated 650 μm microcapsules were then added to a solution of approximately 1.7% (w/v) sodium alginate at a ratio of 1:4 (microcapsules to alginate). This composition was passed through an 18 G airjet into a 1.5% (w/v) buffered CaCl₂ solution using a syringe pump at a flow rate of from 0.6 ml/min to 0.8 ml/min. Nitrogen gas, flowing at a rate of approximately 3 l/min was introduced to shear off the droplet at the desired size. The resulting microcapsule-containing droplets were allowed to polymerize in CaCl₂ for four minutes to form 1100 μm diameter microreactors. The 1100 μm diameter microreactors were then washed three times with media to remove all traces of CaCl₂.

The 1100 μm diameter microreactors were implanted in 41 streptozotocin-induced diabetic C57-BL/6 mice in an amount of from 0.3 ml to 0.6 ml per mouse, which corresponded to about 2500 to about 10,000 islets/mouse.

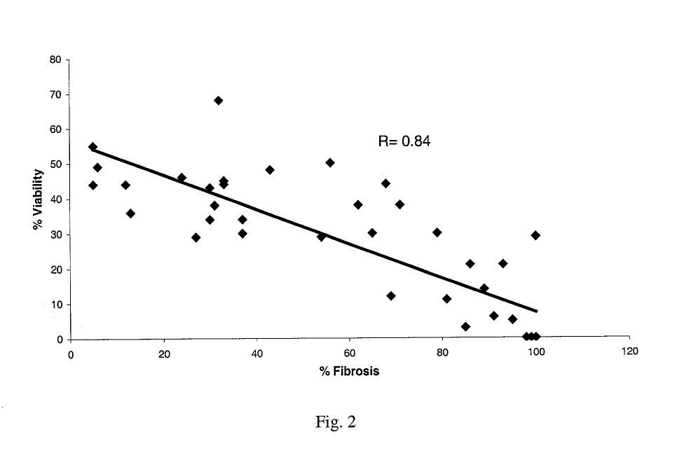

The microreactors were explanted at various points from 7 to 95 days after implantation and analyzed for fibrosis and islet viability using Neutral Red analysis. The results are reported in FIG. 2, which shows a plot of the % viability versus the % fibrosis exhibited by the microreactors.

### Example 1

Microcapsules having a diameter of 3000 μm were prepared by adding porcine islets to an aqueous solution of approximately 1.7% (w/v) sodium alginate in an amount of 45,000 canine islets/ml 1.7% (w/v) sodium alginate from a 14 G airjet into a 1.5% (w/v) buffered CaCl₂ solution using a syringe pump at a flow rate of 0.6 ml/min. Nitrogen gas was introduced to shear off the droplet at the desired droplet size. The droplets were polymerized in the CaCl₂ solution for eight minutes to form microcapsules. The microcapsules were then washed three times with media to remove all traces of CaCl₂.

The 3000 μm microcapsules were then coated with 0.2% (w/v) PLL.

The PLL-coated 3000 μm microcapsules were added to an aqueous solution of approximately 1.7% (w/v) sodium alginate at a microcapsule to 1.7% (w/v) sodium alginate ratio of 1:10. This solution was pipetted through a wide-tip 10 ml pipet (Falcon #7504) into a 1.5% (w/v) buffered CaCl₂ solution. The droplets were allowed to polymerize in the CaCl₂ solution for 16 min whereupon they formed 5000 μm diameter microreactors. The 5000 μm microreactors were then washed extensively with media to remove all traces of CaCl₂.

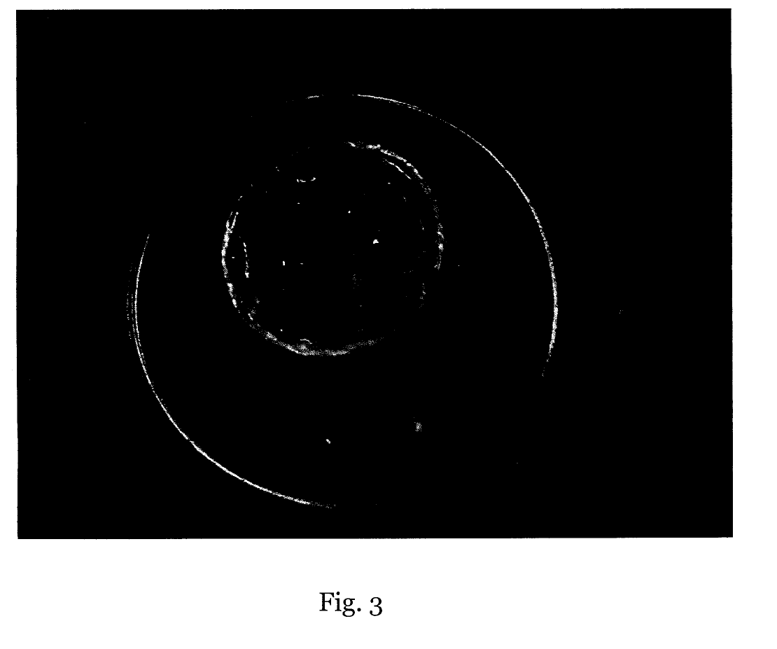

A volume of 5000 μm microreactors was viewed under a microscope. Microreactors having one to four centrally located microcapsules were selected from the volume of microreactors and subsequently implanted in twenty-four C57-BL/6 mice. The microreactors were removed at Day 14. The explanted microreactors were analyzed for % fibrosis using 0.0004% (w/v) Neutral Red and exhibited 18%±6% (Mean±SEM) fibrosis. FIG. 3 is a color photograph of the stained microreactor as seen under a microscope. The red dots are living agents.

### Example 2

Human islets were shipped at room temperature to the facility. The islets were centrifuged at 800 rpm for five minutes and formed a pellet at the bottom of the test tube. The pellet was immediately placed in CRML-1066 culture media lot #11530-037 (Gibco Life Technologies, Grand Island, N.Y.). The number of islets and purity of the islets was determined.

A number of 5000 μm diameter microreactors were prepared as described in Example 1 with the exception that the islets were human instead of canine, the islets were present in an amount of 18,500 human islets/ml 1.7% (w/v) sodium alginate, and the 3000 μm microcapsules were coated with 0.05% (w/v) PLL instead of 0.2% (w/v) PLL.

A volume of 5000 μm microreactors was viewed under a microscope. Microreactors having one to four centrally located microcapsules were selected from the volume of microreactors and subsequently implanted in two STZ induced C57-BL/6 diabetic mice according to the mouse Implantation of Encapsulated Islets method. Each mouse received 12 microreactors containing a total of 19 microcapsules, which corresponded to about 4500 islets.

A second volume of 5000 μm microreactors was prepared as described in Example 1 with the exception that the 3000 μm microcapsules were prepared from a composition of 10,000 porcine islets/ml 1.7% (w/v) alginate and the 3000 μm microcapsules were coated with 0.05% (w/v) PLL.

The 5000 μm microreactors were viewed under a microscope. Microreactors having one centrally located microcapsule were selected from the volume of microreactors and subsequently implanted in the peritoneal cavity of a STZ induced C57-BL/6 diabetic mouse according to the Mouse Encapsulated Islet Implantation method. The mouse received 12 microreactors representing 4,500 islets.

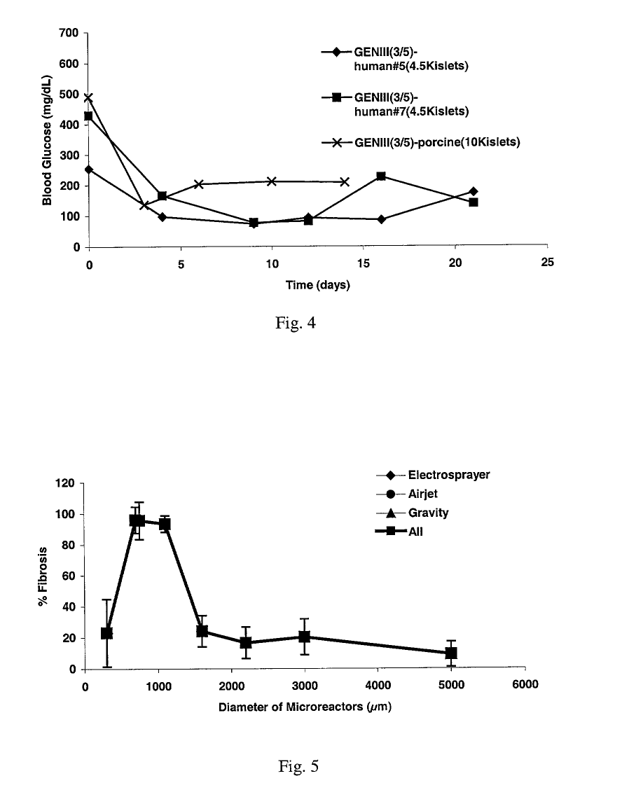

Fasting blood glucose levels were measured every three or four days, approximately. The results are reported in FIG. 4 in the form of a plot of blood glucose level (mg/dL) versus time in days.

After removal, the microreactors were stained with Neutral Red. The microreactors were viewed under a microscope and assayed for fibrosis using the Neutral Red staining method. The microreactors a fibrosis level of 15%.

### Example 3

Alginate spheres, 320 μm in diameter, were prepared by electrosprayer as follows. An aqueous solution of 1.7% (w/v) sodium alginate having a viscosity of 259 centipoise was pumped through a 26 G needle by means of a syringe pump at a rate of 0.2 ml/min. A 17.3 kV/cm DC voltage was applied between the needle and a 1.5% (w/v) CaCl₂ solution. This caused the droplet of alginate solution to separate from the tip of the needle and fall into the buffered 1.5% (w/v) CaCl₂ solution, in which the droplet formed an alginate sphere through ionotropic gelation.

Alginate spheres with diameters of 750 μm, 1600 μm and 2200 μm were also made using an electrosprayer. The 1.7% (w/v) sodium alginate solution was pumped through a 22 G needle using a syringe pump at a flow rate of 1.0 ml/min. The voltage applied to sever a droplet from the tip of the needle was 7.87 kV/cm, 6.3 kV/cm and 5.2 kV/cm, to form the 750 μm, 1600 μm and 2200 μm spheres, respectively.

Alginate spheres having diameters of 700 μm, 1100 μm, and 3000 μm, were prepared by pumping an aqueous solution of approximately 1.7% (w/v) sodium alginate, using a syringe pump at a rate of from 0.6 ml/min to 0.7 ml/min, through 22 G, 18 G and 14 G needles to form the 700 μm, 1100 μm, and 3000 μm spheres, respectively. A co-axial stream of forced nitrogen, at flow rates of 4.8 l/min, 3.0 l/min and 3.0 l/min, which correspond to the 700 μm, 1100 μm, and 3000 μm diameter spheres, respectively, sheared off droplets from the tip of the needle. The droplets fell into a buffered 1.5% (w/v) CaCl₂ solution to form capsules by ionotropic gelation.

Alginate spheres, 5000 μm in diameter, were prepared by gravity by pipetting an approximately 1.7% (w/v) sodium alginate solution through a wide-tip 10 ml pipet into a 1.5% (w/v) buffered CaCl₂ solution. The droplets were allowed to polymerize in the CaCl₂ solution for 16 min whereupon they formed 5000 μm diameter microspheres. The 5000 μm microspheres were then washed extensively with media to remove all traces of CaCl₂.

Four ml of alginate was used to prepare spheres of each of the diameters described above. The final volumes of spheres of each diameter were divided into 8 equal parts and each part was implanted in from 7 to 16 mice for a total of 97 mice so that the total volume and weight of alginate remained constant. Spheres were explanted at 14 to 18 days and assayed for % fibrosis using Neutral Red. The results are reported in FIG. 5 in the form of a plot of % fibrosis versus microreactor diameter in μm.

Spheres ranging in size from 700 μm to 1100 μm produce the highest level of fibrosis in vivo regardless of the method of manufacture. As the diameter of the spheres increased, the amount of fibrosis decreased.

### Example 4

Alginate spheres, 320 μm in diameter, were prepared by electrosprayer in which an approximately 1.7% (w/v) alginate solution having a viscosity of 259 cps was pumped through a 26 G needle by means of a syringe pump at a rate of 0.2 ml/min. A 17.3 kV/cm DC electric field was applied between the needle and a 1.5% (w/v) CaCl₂ solution, which caused the release of alginate droplets into the CaCl₂ gelling solution.

Alginate spheres having diameters of 700 μm, 1100 μm and 3000 μm were prepared using an airjet in which alginate was pumped through an airjet having a needle gauge of 22G, 18G and 14G, respectively, by a syringe pump at a flow rate of from 0.6 ml/min to 0.7 ml/min. A co-axial stream of forced nitrogen at approximate flow rates of 4.8 l/min, 3.0 l/min and 3.0 l/min, respectively, sheared off droplets from the tip of the needle, whereupon the droplets fell into a buffered 1.5% (w/v) CaCl₂ to form alginate spheres by ionotropic gelation.

Alginate spheres, 5000 μm in diameter, were prepared by gravity by pipetting an approximately 1.7% (w/v) sodium alginate solution through a wide-tip 10 ml pipet into a 1.5% (w/v) buffered CaCl₂ solution. The droplets are allowed to polymerize in the CaCl₂ solution for 16 min whereupon they formed 5000 μm diameter spheres. The 5000 μm spheres are then washed extensively with media to remove all traces of CaCl₂.

The above-described alginate spheres (320 μm, 700 μm, 1100 μm, 3000 μm and 5000 μm) were implanted in C57 BL/6 mice, such that the total surface area of implanted spheres remained constant for each sphere diameter implanted. Spheres of each diameter were implanted in seven to eight mice each for a total of 39 mice. The volume of spheres implanted was 51 μl/mouse (for 300 μm spheres), 83 μl/mouse (for 700 μm spheres), 188 μl/mouse (for 1100 μm spheres), 810 μl/mouse (for 3000 μm spheres) and 13 spheres/mouse (for 5000 μm spheres).

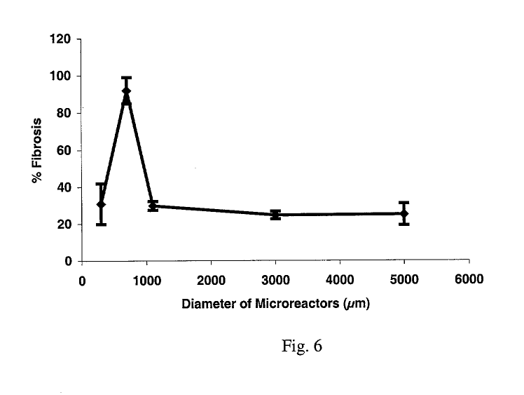

The results are reported in FIG. 6 in the form of a plot of % fibrosis versus microreactor diameter in μm.

Other embodiments are within the claims. Although the microreactor is preferably spherical the microreactor can be of a variety of shapes and preferably is of a shape that maximizes the surface to volume ratio of the microreactor. Examples of other suitable microreactor shapes include cylindrical, oblong, parabolic, hourglass, rhombohedral, discs, and sheets.

What is claimed is:

1. A microreactor comprising:
a. a microcapsule having an average diameter greater than 580 μm, said microcapsule comprising a first matrix and a living agent disposed in said first matrix;
b. a semipermeable membrane surrounding said microcapsule; and
c. a second matrix surrounding said semipermeable membrane, the average distance from said semipermeable membrane to the exterior surface of said second matrix being at least 600 μm.

2. The microreactor of claim 1, wherein said microcapsule is essentially centrally located in said second matrix.

3. The microreactor of claim 1, wherein the average distance from said semipermeable membrane to the exterior surface of said second matrix is at least about 700 μm.

4. The microreactor of claim 1, wherein the average distance from said semipermeable membrane to the exterior surface of said second matrix is at least about 800 μm.

5. The microreactor of claim 1, wherein the average distance from said semipermeable membrane to the exterior surface of said second matrix is at least about 1000 μm.

6. The microreactor of claim 1, wherein said microreactor has an average diameter of at least about 2000 μm.

7. The microreactor of claim 1, wherein said microreactor has an average diameter of at least about 3000 μm.

8. The microreactor of claim 1, wherein said microreactor has an average diameter of at least about 4000 μm.

9. The microreactor of claim 1, wherein said microreactor has an average diameter of at least about 5000 μm.

10. The microreactor of claim 1, wherein said microcapsule has an average diameter of at least about 600 μm.

11. The microreactor of claim 1, wherein said microcapsule has an average diameter of at least about 700 μm.

12. The microreactor of claim 1, wherein said microcapsule has an average diameter of at least about 800 μm.

13. The microreactor of claim 1, wherein said microcapsule has an average diameter of at least about 1000 μm.

14. The microreactor of claim 1, wherein said microcapsule has an average diameter of at least about 2000 μm.

15. The microreactor of claim 1, wherein said microcapsule has an average diameter of at least about 3000 μm.

16. The microreactor of claim 1, wherein said living cell comprises an islet cell.

17. The microreactor of claim 1, wherein said living cell is selected from the group consisting of porcine islet cells, canine islet cells, bovine islet cells, ovine islet cells, human islet cells, non-human primate islet cells, and combinations thereof.

18. The microreactor of claim 1, wherein said semipermeable membrane comprises polyamino acid.

19. The microreactor of claim 18, wherein said polyamino acid is selected from the group consisting of polylysine, polyornithine, polyarginine, polyhistidine and combinations thereof.

20. A method of treating a mammal comprising implanting at least one microreactor in the mammal, said microreactor comprising
a. a microcapsule comprising a first matrix and a living agent disposed in said first matrix, said microcapsule having an average diameter greater than 580 μm,
b. a semipermeable membrane surrounding said microcapsule, and
c. a second matrix surrounding said semipermeable membrane, the average distance from said semipermeable membrane to the exterior surface of said microcapsule being at least 600 um.

21. The method of claim 20, wherein said implanted microreactor is free of an amount of fibrosis lethal to said living agent for at least 14 days.

22. The method of claim 20, wherein said implanted microreactor exhibits no greater than 25% fibrosis for a period of at least 14 days.

23. The method of claim 20, wherein said implanted microreactor exhibits no greater than 20% fibrosis for a period of at least 14 days.

24. The method of claim 20, wherein said implanted microreactor exhibits no greater than 10% fibrosis for a period of at least 14 days.

25. A microreactor comprising:
a. a microcapsule comprising a first matrix and a living agent disposed in said first matrix;
b. a semipermeable membrane surrounding said microcapsule; and
c. a second matrix surrounding said semipermeable membrane, said microreactor, when implanted a higher order mammal for at least 14 days, exhibiting no greater than 25% fibrosis.

26. The microreactor of claim 25, wherein said mammal is a human being.

27. The microreactor of claim 25, wherein said mammal is selected from the group consisting of dogs, cats, horses, cows, pigs and non-human primates.

28. The microreactor of claim 25, wherein said microcapsule has an average diameter of at least 580 μm.

29. The microreactor of claim 25, wherein said microreactor has an average diameter of at least 1500 μm.

30. The microreactor of claim 25, wherein said semipermeable membrane comprises polyamino acid.

31. The microreactor of claim 25, wherein said semipermeable membrane comprises poly-L-lysine, polyornithine, or a combination thereof.

32. The method of claim 25, wherein said microreactor, when implanted in a higher order mammal for at least 14 days, exhibits no greater than 20% fibrosis.

33. The method of claim 25, wherein said microreactor, when implanted in a higher order mammal for at least 14 days, exhibits no greater than 10% fibrosis.

34. A microreactor comprising:
a microcapsule;
a semipermeable membrane surrounding said microcapsule, said semipermeable membrane comprising a component capable of eliciting a fibrotic response from a higher order mammal; and
a second matrix surrounding said semipermeable membrane,
said second matrix being of a thickness sufficient to prevent nucleation of fibrosis by said semipermeable membrane when said microreactor is implanted in a higher order mammal for at least 14 days.

35. The microreactor of claim 34, wherein said mammal is a human being.

36. The microreactor of claim 34, wherein said mammal is selected from the group consisting of dogs, cats, horses, cows, pigs and non-human primates.

37. The microreactor of claim 34, wherein said microcapsule has a diameter of at least 580 μm.

38. The microreactor of claim 34, wherein said second matrix has a thickness of at least 1500 μm.

39. The microreactor of claim 34, wherein said semipermeable membrane comprises polyamino acid.

40. The microreactor of claim 34, wherein said semipermeable membrane comprises poly-L-lysine, polyornithine, or a combination thereof.

41. A method of identifying a microreactor suitable for implantation in a predetermined mammal, said method comprising:
implanting at least one microcapsule in a plurality of the predetermined mammal, the average diameter of the at least one microcapsule implanted in the mammals of one set of mammals being different from the average diameter of the at least one microcapsule implanted in the mammals of at least one other set of mammals;
explanting said microcapsules from the mammals after a period predetermined to be sufficient to elicit a fibrotic response;
analyzing the amount of fibrosis present on the surface of the explanted microcapsules;
determining whether the microcapsules of a set exhibit an average of no greater than 25% fibrosis,
if such a microcapsule is present, identifying the average diameter of the microcapsule as being suitable for implantation in the mammal; and
if such a microcapsule is not present, repeating said implanting, explanting, analyzing and determining until a microcapsule exhibiting no greater than 25% fibrosis is present, and identifying the average diameter of said microcapsule.

42. The method of claim 41, wherein said determining comprises determining whether at least one microcapsule exhibits no greater than 20% fibrosis.

43. The method of claim 41, wherein said determining comprises determining whether at least one microcapsule exhibits no greater than 10% fibrosis.

44. The method of claim 41, wherein said determining comprises determining whether at least one microcapsule exhibits no greater than 5% fibrosis.

45. The method of claim 41, wherein the volume of microcapsules implanted in each mammal is essentially the same.

46. The method of claim 41, wherein the total surface area of the microcapsules implanted each mammal is essentially the same.

47. A method of treating a mammal comprising implanting a microreactor having a predetermined diameter in the mammal, said microreactor diameter having been predetermined for the mammal according to the method of claim 41.

48. A microreactor suitable for implantation in a predetermined mammal, said microreactor comprising:
a microcapsule comprising a matrix and a living agent disposed in said matrix;
a semipermeable membrane surrounding said microcapsule; and
a second matrix surrounding said semipermeable membrane,
the suitability of said microreactor for said predetermined mammal having been predetermined according to the method of claim 41.

49. A method of identifying a microreactor suitable for implantation in a predetermined mammal, said method comprising:
implanting at least one microreactor in a plurality of the predetermined mammal, said microreactor comprising a microcapsule, a semipermeable membrane surrounding said microcapsule and a second matrix surrounding said semipermeable membrane, the composition of said semipermeable membrane being capable of eliciting a fibrotic response from the predetermined mammal,
the second matrix thickness of the microreactors implanted in the mammals of one set of mammals being different from the second matrix thickness of the microreactors implanted in the mammals of at least one other set of mammals;
explanting said microreactors from said mammals after a period predetermined to be sufficient to elicit a fibrotic response to a foreign body from the predetermined mammal;
determining whether the microreactors of a set are, on average, free of nucleated fibrosis; and,
if a microreactor is free of nucleated fibrosis, identifying the thickness of the second matrix of the microreactor as being suitable for implantation in the predetermined mammal,
if a microreactor exhibits nucleated fibrosis, repeating said implanting, explanting and determining until a second matrix thickness suitable for implantation in the predetermined mammal is identified.

50. A method of treating a mammal comprising implanting a microreactor in a mammal, said microreactor comprising a second matrix having a thickness predetermined for said mammal according to the method of claim 49.

51. The method of claim 49, wherein said mammal is a human being.

52. The method of claim 49, wherein said microreactor comprises a microcapsule, a semipermeable membrane surrounding said microcapsule, and a second matrix surrounding said semipermeable membrane.

53. The method of claim 49, wherein said microcapsule is substantially centrally located in said microreactor.

54. The method of claim 49, wherein said mammal is diabetic and dependent upon a source of insulin exogenous to said mammal, said method further comprising implanting said microreactors in an amount sufficient to achieve a reduction in said mammal's dependence on exogenous insulin.

55. The method of claim 49, wherein said mammal is diabetic, said method further comprising implanting said microreactors in an amount sufficient to achieve glucose homeostasis in said mammal.

56. A method of identifying a microreactor suitable for implantation in a predetermined mammal, said microreactor comprising a microcapsule, a semipermeable membrane surrounding said microcapsule and a second matrix surrounding said semipermeable membrane, said method comprising performing the method of claim 41 and performing the method of claim 49.

57. A microreactor suitable for implantation in a predetermined mammal, said microreactor comprising:
a microcapsule comprising a first matrix and a living agent disposed in said first matrix;
a semipermeable membrane surrounding said microcapsule; and
a second matrix surrounding said semipermeable membrane,
the suitability of said microreactor having been predetermined for the mammal according to the method of claim 41.

58. A microreactor suitable for implantation in a predetermined mammal, said microreactor comprising:
a microcapsule comprising a matrix and a living agent disposed in said matrix;
a semipermeable membrane surrounding said microcapsule; and
a second matrix surrounding said semipermeable membrane,
the suitability of said microreactor having been predetermined for the mammal according to the method of claim 49.

59. A method of selecting a microreactor for implantation in a mammal, said method comprising:
a. identifying a microreactor comprising
i. a microcapsule said microcapsule comprising
a first matrix,
a living agent disposed in said first matrix, and
a semipermeable membrane surrounding said first matrix, and
ii. a second matrix surrounding said semipermeable membrane, said microcapsule being essentially centrally located in said second matrix, the average distance between the exterior surface of said microcapsule and the exterior surface of said second matrix being at least 600 um,
b. removing said microreactor from a plurality of microreactors.

60. The method of claim 59, wherein said microreactor is removed from a plurality of microreactors prior to identifying said microreactor.

61. The method of claim 59, wherein said microreactor is removed from said plurality of microreactors subsequent to identifying said microreactor.
